(19) United States
(12) Patent Application Publication
(10) Pub. No.: US 2010/0204551 A1
(43) Pub. Date: Aug. 12, 2010

(54) Detection, Prevention and Treatment of Infections in Implantable Devices

(76) Inventor: Martin William Roche, Fort Lauderdale, FL (US)

Correspondence Address: Orthosensor, Inc., 1560 Sawgrass Corporate Pkwy, 4th Floor, Sunrise, FL, 33323, US

(21) Appl. No.: 12/604,083

(22) Filed: Oct. 22, 2009

Related U.S. Application Data
(60) Provisional application No. 61/196,915, filed on Oct. 22, 2008.

(51) Int. Cl. A61B 5/00; A61N 1/00

(52) U.S. Cl. 600/301; 607/116

(57) ABSTRACT

At least one embodiment is directed to a system (400) to detect a presence of bacteria or other infecting organism in proximity to an implanted device. The system (400) comprises one or more biological sensors (412, 414, 416, 418) a processing unit (420), and a screen (422). Biological sensors (412, 414, 416, 418) detect a presence of bacteria or infecting organisms. Once an infection is detected, the system (400) can activate the release of anti-infective elements local to the implanted device. In one embodiment, nanostructures are used to retain the anti-infective elements until needed. A pulsed electrical field is applied in infected regions proximal to the implanted device. The pulsed electric field initiates electroporation allowing increased cell wall penetration of the anti-infective elements. The system (400) responds to an infection after surgical implantation and eradicates bacteria without the need for surgical intervention or implant removal.

### CROSS-REFERENCE TO RELATED APPLICATIONS

This application claims the priority benefits of U.S. Provisional Patent Application No. 61/196,915, U.S. Provisional Patent Application No. 61/196,914, and U.S. Provisional Patent Application No. 61/196,916 all filed on Oct. 22, 2008, the entire contents of which are hereby incorporated by reference.

### FIELD

The invention relates in general to implantable devices in living organisms, and particularly though not exclusively, is related to the detection and treatment of infections in and around an implantable device.

### BACKGROUND

Implantable devices are becoming more prevalent. Complex mechanical and electrical systems such as pacemakers, heart defibrillators, orthopedic implants, neurological devices are but a few of the systems being implanted on a common basis. Implantable devices have proven reliable and are placed inside the human body for extended periods. As one example, an orthopedic implant can be used to repair a damaged joint of the human skeletal system. Surgery is generally invasive and requires one or more incisions to access the joint region. Furthermore, in a complete joint replacement, bone is cut in the joint region and the articulating surfaces of the joint are replaced. The artificial joint typically comprises light weight metals and high strength polymers. Movement is still enabled by muscle tissue and tendons attached to the skeletal system around the artificial joint. Ligaments hold and stabilize the one or more joint bones positionally.

In general, post-operative care after implant surgery typically includes periodic doctor visits. A tentative approach is often taken to determine if complications or difficulties arise, although medicine and therapy is often prescribed. Much of this approach relies on the patient to provide feedback to the physician, surgeon, or caretaker should anything out of the ordinary arise. A common problem with an implanted device, such as an orthopedic implant, is that the patient may be unaware of a serious infection or problem that is occurring. By the time the patient identifies the problem it may have already escalated to a significant health risk potentially leading to catastrophic consequences.

FIG. 1 is an illustration of components of a hip prosthesis 100 as known in the art. A hip replacement typically comprises a cup 110, a bearing 112, and a femoral implant 106. In at least one arrangement, cup 110 comprises metal or other material of high strength. The cup 110 is fitted and attached to the acetabulum of a pelvis 102. A bearing 112 is fitted into cup 110 for providing a low friction low wear surface in which a femoral head 108 of femoral implant 106 is fitted. Bearing 112 typically comprises a plastic material such as ultra high molecular weight polyethylene. In general, a predetermined amount of surface area of femoral head 108 is in contact with the surface of bearing 112 to minimize loading and wear on the material. Femoral implant 106 is fastened into a proximal end of femur 104 of the lower leg. Femoral implant 106 comprises a strong lightweight material and typically comprises a metal or metal alloy. Portions of all the components of the hip replacement are exposed internally to the patient. The hip replacement components are selected to be formed of biologically compatible materials.

FIG. 2 is an illustration of components of a knee prosthesis 200 as known in the art. Knee prosthesis 200 comprises a femoral implant 208, an insert 210, and a tibial implant 212. A distal end of femur 202 is prepared and receives femoral implant 208. Femoral implant 208 has two condyle surfaces that mimic a natural femur. Femoral implant 208 is typically made of a metal or metal alloy. A proximal end of tibia 204 is prepared to receive tibial implant 212. Tibial implant 212 is a support structure that is fastened to the proximal end of the tibia and is usually made of a metal or metal alloy. Tibial implant 212 also retains insert 210 in place. Insert 210 is fitted between femoral implant 208 and tibial implant 212. Insert 210 has two bearing surfaces in contact with the two condyle surfaces of femoral implant 208 that allow rotation of the lower leg under load. Insert 210 is typically made of a high wear plastic material that minimizes friction.

FIG. 3 is an illustration of a spinal implant 302 as known in the art. Spinal implant 302 is shown between vertebrae of a spinal column. In general, a spinal implant involves a disc region between two vertebrae. Degeneration of a disc can result in irritation of the nerves of the spinal column that results in severe back pain. Spinal fusion is a common method to address the issue by fusing adjacent vertebrae together such that the two vertebrae cannot move in relation to each other. One method of fusing uses a spinal cage. As it name implies a cage is inserted between a vertebrae 304 and a vertebrae 306. The spinal cage is designed to promote bone growth to fixate the two vertebrae in conjunction with the insert. The spinal cage is formed of metal or metal alloy. Typically, a bone graft is required to establish bone growth that fuses the vertebrae together.

Alternatively, replacement discs are being introduced. The replacement or artificial disks comprise an upper plate, a flexible core, and a lower plate. The upper and lower plates are formed from metal or metal alloy and can be fastened to a vertebral surface. In one arrangement, metal spikes or teeth extend from the plate surface that penetrates a vertebral surface holding the artificial disk in place. The flexible core is attached to the upper plate and lower plate of the artificial disk. The flexible core can contain a gel, rubber, foam, or other material that mimics the flexibility and compression capability of a natural disk under compressive and rotative forces.

The orthopedic devices described briefly above are examples of an implanted system that is inserted within the body of an organism. Some surgical procedures are more invasive than others but all are prone to reactions to the device and post operative complications. In particular, infection in the field of orthopedics, cardiac, and neurosurgery continues to cause a significant percentage of morbidity and continued patient care at high cost. The use of implantable devices in these and other fields carries the risk of early as well as late stage sepsis.

A problem associated with implanted devices is that they can be an ideal breeding ground for bacteria. In the examples described above, synovial fluid in combination with the implanted orthopedic device can actually promote bacterial growth. Synovial fluid is a naturally secreted fluid in a joint region. The synovial fluid contains nutrients that can sustain bacteria. An implanted device, for example an orthopedic joint comprises multiple surfaces and interfaces that form areas where bacteria can acclimate and multiply. The problem is further compounded by the fact that the infection often goes unnoticed by the patient. More problematic is that the patient often does not realize that the problem exists until the infection is firmly established resulting in a more difficult recovery. The standard treatment is primarily prophylactic and then treatment with antibiotics and commonly surgical removal of the prosthesis or device once the infection occurs.

### BRIEF DESCRIPTION OF THE DRAWINGS

Exemplary embodiments of present invention will become more fully understood from the detailed description and the accompanying drawings, wherein:

FIG. 1 is an illustration of components of a hip prosthesis as known in the art;

FIG. 2 is an illustration of components of a knee prosthesis as known in the art;

FIG. 3 is an illustration of a spinal implant as known in the art;

FIG. 4 is an illustration of a system for preventing infection on an implanted device in accordance with an exemplary embodiment;

FIG. 5 is an illustration of an implanted device having bacteria in synovial fluid around the artificial joint;

FIG. 6 is an illustration of a pulsed electric field emitted in proximity to an implanted device in accordance with an exemplary embodiment;

FIG. 7 is an illustration of bacterial response to a field in proximity to an implanted device in accordance with an exemplary embodiment; and

FIG. 8 depicts an exemplary diagrammatic representation of a machine in the form of a computer system within which a set of instructions, when executed, may cause the machine to perform any one or more of the methodologies disclosed herein.

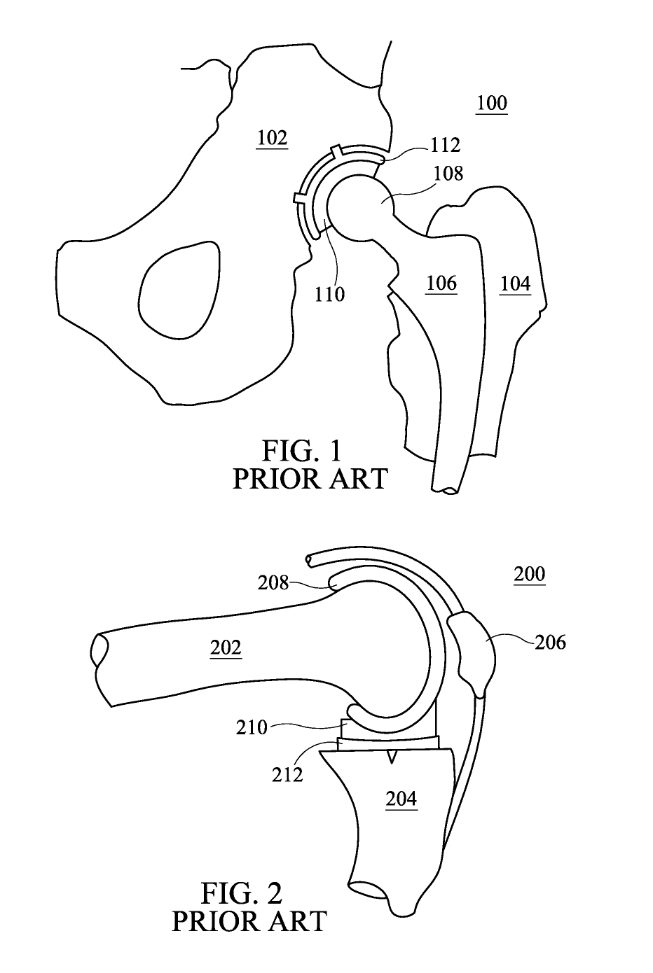

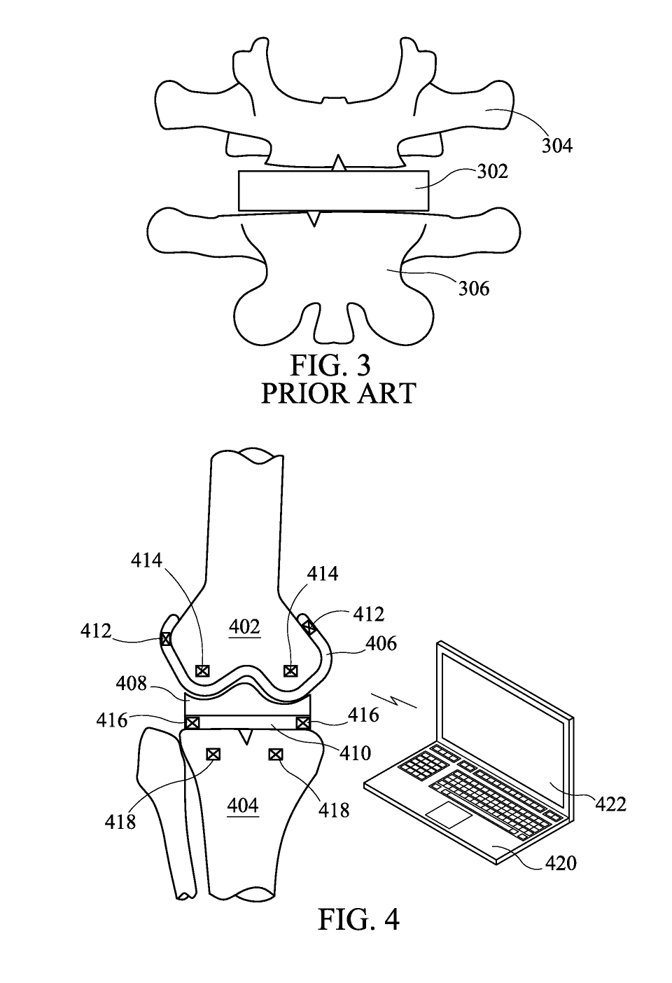

### DETAILED DESCRIPTION

The following description of exemplary embodiment(s) is merely illustrative in nature and is in no way intended to limit the invention, its application, or uses.

Processes, techniques, apparatus, and materials as known by one of ordinary skill in the art may not be discussed in detail but are intended to be part of the enabling description where appropriate. For example specific computer code may not be listed for achieving each of the steps discussed, however one of ordinary skill would be able, without undo experimentation, to write such code given the enabling disclosure herein. Such code is intended to fall within the scope of at least one exemplary embodiment.

Additionally, the sizes of structures used in exemplary embodiments are not limited by any discussion herein (e.g., the sizes of structures can be macro (centimeter, meter, and size), micro (micro meter), nanometer size and smaller).

Notice that similar reference numerals and letters refer to similar items in the following figures, and thus once an item is defined in one figure, it may not be discussed or further defined in the following figures.

In all of the examples illustrated and discussed herein, any specific values, should be interpreted to be illustrative only and non-limiting. Thus, other examples of the exemplary embodiments could have different values.

In general, the successful implantation of a device in an organism and more specifically in a joint or spine depends on multiple factors. One factor is that the surgeon strives to implant the device to obtain adequate alignment of the extremity or spine. A second factor is proper seating of the implant for stability. A third factor is that orthopedic implants typically comprise more than one component that are aligned in relation to one another. A fourth factor is balance of loading over a range motion. A fifth factor and a more general factor that relates to all implanted devices is to minimize infections that can occur post-operatively.

In a first embodiment a system includes an implantable device and a biological sensor coupled to the implanted device. The biological sensor is exposed to the interior of the organism to detect a presence of bacteria and other infecting organisms in proximity to the implantable device post-operatively, for example, after the device is implanted. The system can identify potential medical problems early after surgical implantation of the implantable device and take appropriate measures upon identification of the problem. Benefits of this early diagnosis may reduce post operative rework with substantial benefits in lowering invasive post-operative procedures, decreasing cost, freeing up operating rooms, and minimizing patient stress.

In a second embodiment, a system includes an implantable device having a major surface interior to an organism, a first and second electrode where a portion of the interior of the organism is between the first and second electrodes, and a pulsing circuit operatively coupled to the first and second electrode. Each pulse from the pulsing circuit generates an electric field between the first and second electrodes. The electric field electroporates one or more cells of bacteria or an infecting organism in proximity to the generated electric field. The system can control a level and delivery of a pharmaceutical agent during the electroporation.

In a third embodiment, a system includes an orthopedic joint implant where a portion of the major surface has a plurality of nanostructures coupled thereto, a biosensor to detect a presence of bacteria or infecting organisms, and a control circuit operatively coupled to the at least one biosensor and the nanostructures to enable a release of the agents contained in the nanostructures. The nanostructures include agents, hydrogels, antibiotics, or cytoxins to reduce infection by bacteria or an infecting organism and prevent bacterial growth in a joint region.

Several implant devices were briefly described earlier, each of which can be configured in accordance with the embodiments herein above. More specifically, orthopedic devices are shown because they typically comprise multiple components that have multiple surfaces internal to a patient. It should be noted that orthopedic devices are used for illustrative purposes. Various embodiments herein apply to devices implanted internal to an organism. Other examples of implantable devices are monitoring devices, drug delivery devices, pace makers, defibrillators, to name but a few. A common factor in implanted devices is that post-operative infections can occur and that the device itself can enable the bacteria or infecting organism to thrive.

FIG. 4 is an illustration of a system 400 for preventing infection on an implanted device in accordance with an exemplary embodiment. The implantable device can be used in hip, knee or spine prosthetics or other orthopedic joints as previously described and shown. A platform to monitor and react to an early or late infection is described hereinbelow. In particular, the platform can detect infections in an early stage where if detected can be treated effectively to eliminate the problem. Detection further eliminates an issue where the patient with an implant is often unaware of an infection and does not seek help until the bacteria or infecting organism is firmly established. System 400 also addresses a problem with the implant itself. The implanted device in conjunction with the local biology can provide areas that can harbor, provide sustenance and fuel growth of the infection.

In at least one exemplary embodiment, system 400 includes one or more sensors that will identify an early infection before it becomes chronic, seeds the device, and prevents the penetration of antibiotics. Most device implants are made of metal or plastics that can be coated by the bacteria allowing them to multiply. In a non-limiting example, a knee implant is used to illustrate the system. The system can be applied to other implanted devices or systems. The knee implant comprises a femoral implant 406, an insert 408, and a tibial implant 410. Femoral implant 406 is coupled to a distal end of femur 402. Similarly, tibial implant 410 is coupled to a proximal end of tibia 404. An insert 408 is coupled between femoral implant 406 and tibial implant 410. Insert 408 provides a bearing surface on which the condyles of femoral implant 406 contact allowing rotation of the lower leg. In general, at least one biological sensor is coupled to the implanted device such as a knee implant. Typically, more than one biological sensor is used to detect bacteria or an infecting organism in a region in and around the implanted device. Note that infection has the highest probability of occurring within a relatively short period of time following the surgical procedure. Moreover, the highest concentration of bacteria will most likely occur in the vicinity of the implant for the reasons discussed above.

As shown, multiple sensors are used to determine if bacteria is present in proximity to the knee implants. In at least one exemplary embodiment, sensors for detecting the presence are placed in a variety of locations near the knee implant. Bacteria is detected in proximity to the distal end femur 402 by sensors 412 that are in and part of femoral implant 406. Further coverage of the distal end of femur 402 is obtained by sensors 414 placed in or attached to the distal end of femur 414. Similarly, bacteria is detected in proximity to the proximal end of tibia 404 by sensors 416 that are in and part of tibial implant 410. Additional coverage is achieved by sensors 418 placed in or attached to the proximal end of tibia 404. Sensors 412 and 416 also detect a presence of bacteria between femoral implant 406 and tibial implant 410.

Different methods can be used to determine if an infection is present. The biological sensors 412, 414, 416, and 418 can detect bacteria or other infecting organism by measuring parameters in proximity to the implanted devices such as pH, temperature, viscosity, blood flow, a change in material property corresponding to a change in frequency, and by the detection of cell wall markers. For example, the most prevalent bacteria causing post-operative infections in an implanted joint are the staphylococcus bacteria. In the non-limiting example, synovial fluid around the joint can be monitored by sensors 412, 414, 416, and 418. Non-infected synovial fluid will be within a predetermined range of pH, temperature, viscosity. Measuring parameters outside the predetermined range can indicate the presence of an infection. A differential analysis can also be used. The synovial fluid can be monitored immediately after the orthopedic device is implanted. The measured parameters are then monitored for changes. A significant change in a measured parameter or a change in combination with the absolute measured value can be used to indicate the presence of an infection.

Sensors 412, 414, 416, and 418 can comprise more than one sensor type. A combination of sensors providing more than one measured parameter can be used in the determination of the presence of bacteria or an infecting organism. In at least one embodiment, multiple types of sensors are used in and around the implanted device. A sensor can be a sensor array comprising more than one type of sensor integrated into a common housing. Conversely, separate and different types of sensors can be placed where needed. Measuring more than one parameter can aid in the identification of the type of bacteria present or provide early detection of an onset of an infection. The pH of synovial fluid will turn increasingly acidic in the presence of bacteria such as the staphylococcus bacteria. Thus, exceeding a predetermined pH threshold can trigger (e.g. equal to or lower than the predetermined threshold value) an infection event. Similarly, a change in pH above a predetermined differential value (e.g. a negative change in pH) could also be used to trigger the infection event. The temperature of the synovial fluid will rise with the increasing presence of bacteria in synovial fluid. Thus, exceeding a predetermined temperature or a exceeding a predetermined positive differential change in temperature can be used to trigger the infection event. The viscosity of the synovial fluid will increase in turbidity, as more bacteria are present. Thus, exceeding a predetermined viscosity or exceeding a predetermined change in viscosity can be used to trigger the infection event. The detection of fluid color can also be applied to some applications. For example, synovial fluid is normally a yellow color that turns to a grey color as the bacteria count rises. Monitoring a change in color can be a useful indication of bacteria and start of an infection.

In at least one exemplary embodiment, a signal can be sent through the synovial fluid and the frequency of the signal is monitored over time. In general, a transmitter and receiver are a fixed distance apart. The synovial fluid passes between the transmitter and receiver. Post-operatively, the signal will have a characteristic frequency corresponding to the fluid properties. This characteristic frequency is indicative of a condition where little or no bacteria are present. A build up of bacteria in the synovial fluid will change how the frequency propagates through the fluid. In at least one exemplary embodiment, a change in propagation time results in a change in the frequency. Thus, a change in frequency can be used to determine the presence of bacteria.

Analysis of a bacterial cell wall is a direct method for determining the presence of bacteria and the type of bacteria. In particular, a sensor looks for one or more components of the bacterial cell wall that comprises an identifying marker. For example, resonance can be used to break apart bacterial cell walls. The components of the cell walls or cell wall fragments in the synovial fluid are detected by the sensor. Detecting the presence of the marker indicates an infection. The concentration of the marker can indicate the level of the infection.

A preventative measure can be a local release to the implanted device region of antibiotics, cytotoxins, or other elements to eliminate bacteria and infecting organisms near the joint. The release of the medicine would occur over a predetermined time period shortly after surgery to implant the device. This can be done during the critical post surgical period when infection is likely to occur. Local release of medicine where the infection occurs allows a much lower dose to be used. The implementation will be discussed in more detail hereinbelow. Sensors 412, 414, 416, and 418 can then be used to monitor a region around the implanted device for bacteria although the preventative measures would greatly reduce the likelihood of an infection.

Alternatively, it may not be desirable to release medicine (even locally) unless an infection is imminent. Harmful bacteria are detected when a measured parameter exceeds the predetermined thresholds of sensors 412, 414, 416, and 418. Since bacteria are present, measures are undertaken to suppress or prevent an infection from occurring. One measure is to send a signal that can be transferred to the doctor or patient indicating a problem. The doctor can then prescribe medication to the patient that will eliminate the bacteria or infecting organism before a severe infection occurs. As mentioned above, system 400 can include a response such as antibiotics and cytotoxins that are released in proximity to the joint when infecting bacteria are found to be within range of the sensors.

In at least one exemplary embodiment, sensors 412, 414, 416, and 418 comprise a sensor for measuring a parameter, a control circuit, circuitry for wired or wireless communication, and a power source. The control circuit can be a mixed mode circuit having both analog and digital circuitry. The control circuit is configured operatively to the sensor and communication circuitry to manage when measurements are taken, sending the data for appropriate review, or triggering a local response. In one embodiment, each sensor has a control circuit, communication circuitry, and a power source. Each sensor can be powered by a battery or a temporary power source. Alternatively, a single control circuit can be coupled to sensors 412, 414, 416, and 418 for receiving information from each sensor (wired or wirelessly) and transmitting the measured data to an appropriate client.

In one embodiment, the control circuit includes circuitry to convert the data to a form that can be transmitted by wire or wirelessly. For example, the control circuit can have transmitter/receiver circuitry for transmitting digital or analog data in a standardized communication platform such as Bluetooth, UWB, or Zigbee. In one embodiment, each control circuit enables each sensor to measure data periodically or by command. Furthermore, the measured data can be stored in memory and sent when appropriate thereby preventing information being sent by all sensors simultaneously. A signal can also be generated by each control circuit and sent when a predetermined threshold of sensors 412, 414, 416, and 418 is exceeded.

System 400 further includes processing unit 420 having a screen 422. Processing unit 420 is in communication with sensors 412, 414, 416, and 418. Processing unit 420 can be a digital processing unit, microprocessor, logic circuit, notebook computer, personal computer, or other similar type device. Processing unit 420 can control when sensors 412, 414, 416, and 418 take measurements and send data. Measured parameters from sensors 412, 414, 416, and 418 can be analyzed by processing unit 420 and appropriate actions taken. For example, processing unit 420 can notify the patient that a problem exists, notify the hospital/doctor that an infection has been detected, or take local action by enabling a release of medicine to eliminate the infecting organism (if the action was not taken by the sensors). The data can be displayed on screen 422 to show the parameters measured by each sensor such that the location, severity, and infection type is understood.

As shown, sensors 414 and sensors 418 can be inserted or attached respectively to femur 402 and tibia 404 of the lower leg. For example, sensors 414 and 418 can be placed in a housing that has external screw threads. The sensors in a screw type housing can then be attached in bone using tools common to an orthopedic surgeon. Alternatively, the sensors can be temporarily attached to the bone, an implant device, or a surgical tool so they can be removed or disposed of. For example, a sensor array can be pinned to bone for temporary or permanent use. The sensors can also be incorporated into the implanted device as described hereinabove.

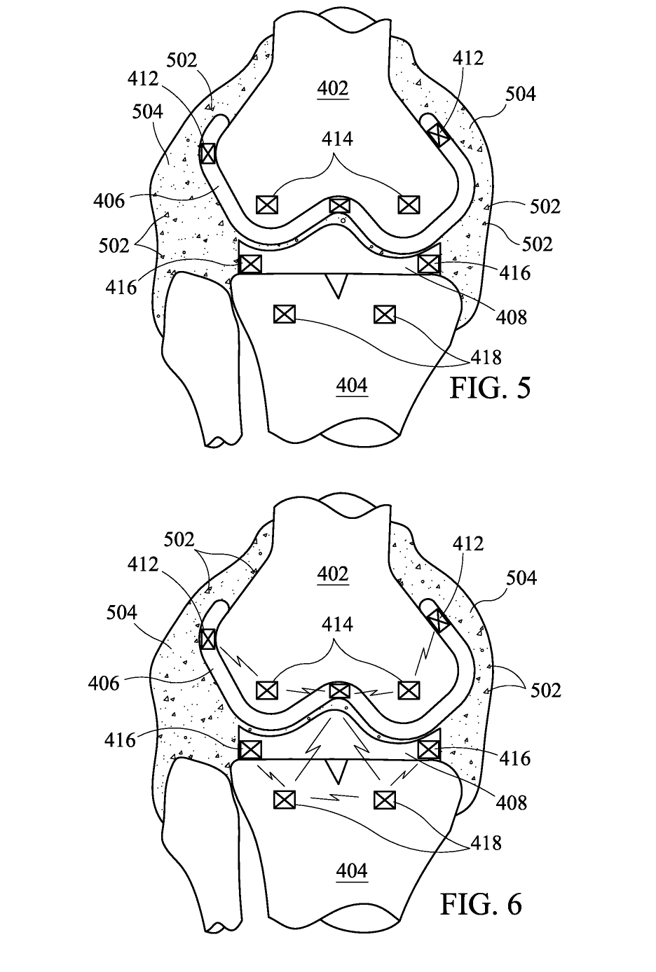

FIG. 5 is an illustration of an implanted device having bacteria in synovial fluid around the artificial joint. A synovial membrane secretes synovial fluid into a joint space around the joint. Synovial fluid is a natural lubricant for the contacting surfaces of an articulating joint. The liquid in combination with the artificial joint create an environment that can sustain and fuel the growth of bacteria. The synovial fluid contains glucose, which bacteria can feed on. The surfaces and interfaces of the artificial joint form areas in which the bacteria can have safe harbor as it multiplies and becomes established which ultimately can lead to sepsis.

FIG. 6 is an illustration of a pulsed electric field emitted in proximity to an implanted device in accordance with an exemplary embodiment. In one embodiment, sensors comprising electrodes for creating a field are placed in proximity to the implanted device. The sensors are activated to generate a pulsed electrical field in the presence of bacteria or an infecting organism. The pulsed electric field induces electroporation, which is the act of applying an electrical field to a cell membrane that raises electrical conductivity and increases the permeability of the cell plasma membrane. Sensor system 400 will activate a pulsed electrical field between two or more of the elements to increase the permeability of bacteria within the field. Sensor system 400 will allow modulation of the pulse electrical amplitude, duration, wave number, waveform, and inter-pulse intervals. The predetermined electrical field strength for a predetermined time period will generate a membrane potential that penetrates the cell wall to be activated. Temperature changes and cellular strength can be monitored during the electroporation process. The weakened cell membrane is made more permeable so that the bacteria can readily receive antibiotics, cytotoxins or other medicine that can eliminate the bacteria or an early stage infection. In at least one exemplary embodiment, the medicine is released locally in proximity to the sensors and the implanted device.

In a non-limiting example, sensors 412, 414, 416, and 418 are electrodes strategically placed to apply an electric field in locations around an implanted knee joint and more specifically across volumes of synovial fluid. Alternatively, a micromachined structure can be used to generate the pulsed electric field. One or more sensors detecting a presence of an infecting bacteria can initiate an electroporation process. A doctor or health care professional could also initiate the process by sending a signal to the control circuits of each sensor. A control circuit can be used to sequence the pulsing of sensors 412, 414, 416, and 418 such that the synovial fluid and thereby the bacteria in proximity to the knee implant, distal end of femur 402, and proximal end of tibia 404 are subject to electroporation. The control circuit is operatively coupled to a pulsing circuit in each sensor for generating a pulsed voltage. A voltage multiplier can be used to provide a voltage not provided by the power source. In at least one exemplary embodiment, an electric field of between 0.2 kV/cm to 20 kV/cm is used to induce electroporation. Pulse duration is typically from microseconds to milliseconds in length. Pulse shape can also effect the amount of permeability achieved and can be tailored for the specific bacteria and application.

In at least one exemplary embodiment, two or more components of the implanted device can be electrodes for the electroporation process. For example, in a knee implant, a major surface (or portion thereof) of femoral implant 406 can be a first electrode. Insert 408 typically comprises a non-conductive material. A second electrode can be embedded in insert 408. Similarly, tibial insert can be an electrode. Bacteria in synovial fluid between and around the implanted devices would be subject to a pulsed electric field.

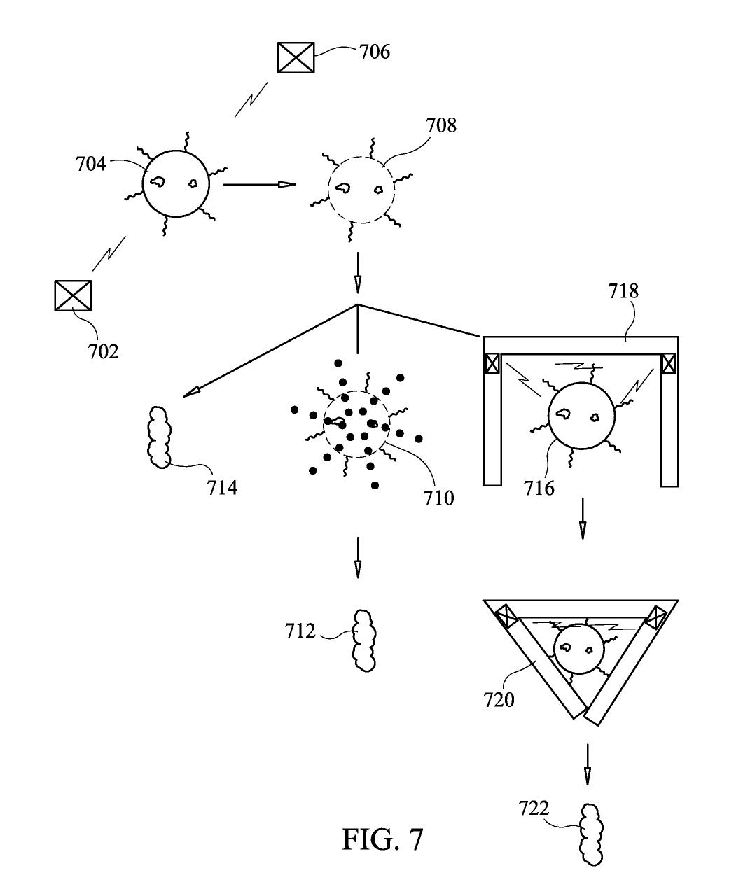

FIG. 7 is an illustration of bacterial response to a field in proximity to an implanted device in accordance with an exemplary embodiment. Sensors 704 and 706 are placed on or in proximity to the implanted device. A bacteria in a first state 702 is between sensors 704 and 706. A pulsed voltage is applied across sensors 704 and 706 creating a momentary electric field. The pulsed electric field disrupts the cell membrane creating cracks or opening pores of the cell wall creating a bacteria in a second state 708. The openings in the cell membrane can be either temporary or permanent. The bacteria in the second state 708 have increased permeability from the first state 702.

The increased permeability of the bacteria in the second state 708 allows the penetration of antibiotics, cytokines, or other medicines that can be absorbed through the cell wall to kill the bacteria. The medicine can be provided to the body by injection, pills, or other common means. In at least one exemplary embodiment, a coating is applied to the implanted device or a portion of the implanted device is made of nanostructures that can house hydrogels, antibiotics, cytotoxins, and other elements that by changing the medium the bacteria live in would cause damage to the organism cell wall. For example, the nanostructures can be attached to exposed surfaces of femoral implant 406, insert 408, and tibial insert 410 in areas exposed to synovial fluid. The nanostructures would be activated by a biosensor to release the anti-infective elements while the pulsed electrical field will potentiate uptake by the infecting organism in a third state 710. Thus, a combination of increased cell wall permeability and local release of medicine to the infected region maximizes delivery into the bacterial cell internal structure. The efficient delivery of the medicine results in a cell death of the bacteria in a fourth state 712. In at least one exemplary embodiment, the biosensor can target different regions of nanostructures to release medicine thereby controlling the concentration over time.

A further application of the pulsed electrical field is to destroy the cell wall membrane resulting in the bacteria in a fifth state 714. In at least one exemplary embodiment, the electric field is pulsed at a resonant frequency of the bacteria. In resonance the energy applied to the cell walls of the bacteria is additive. Resonance destroys the cell wall membrane such that the organism is killed and/or prevented from multiplying. Reducing the level of the infection by resonant destruction of bacteria allow our internal macrophages and lymphocytes to attack the remaining organisms.

As mentioned previously, nanostructures on a surface of the implanted device could contain or be formed from hydrogels. The hydrogel nanostructures can be formed as a compartment having an opening that can receive one or more bacteria. The hydrogel nanostructure can also be made to attract bacteria. For example, the hydrogel can include a chemical that attracts the bacteria. Alternatively, the nanostructure can be polarized or charged to attract the bacteria.

In at least one exemplary embodiment, a bacteria 716 enters an opened nanostructure 718 to trap the infective organism. The hydrogel wall of the nanostructure 718 can be modulated by the biosensors (pH) and the sensors electrical impulses as well as other local mediators. The bacteria 716 is thus identified in nanostructure 718 and the hydrogel walls collapse to contain bacteria 716 in closed nanostructure 720. Bacteria 716 cannot multiply or obtain sustenance while contained in nanostructure 720 and undergoes cell death 722.

By now it should be realized that a substantial benefit is achieved by having a smart implant that recognizes infection; activates the release of anti-infective elements, that will along with the generation of a pulsed electrical field, lead to cell wall penetration and ultimately death of the infecting organism The smart system utilizes bio-sensors, piezo-sensors, micromachined structures, and nanostructures having a small foot print that can be integrated into an implanted device as well as attached to parts of the body. This will allow the earliest response to infection and the potential to eradicate the infection without the need for surgical intervention or implant removal.

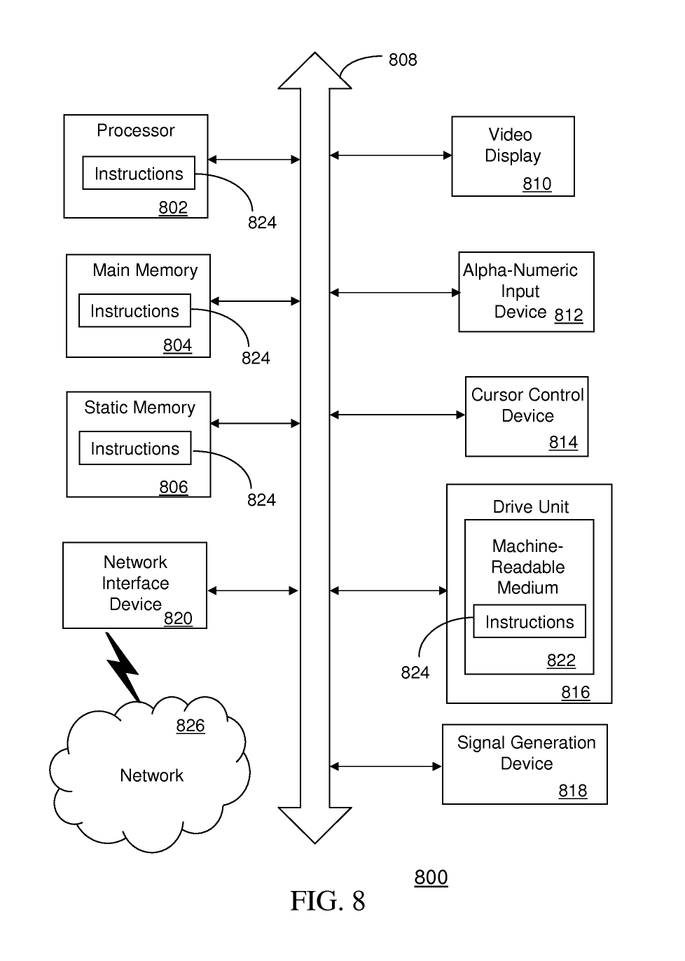

FIG. 8 depicts an exemplary diagrammatic representation of a machine in the form of a computer system 800 within which a set of instructions, when executed, may cause the machine to perform any one or more of the methodologies discussed above. In some embodiments, the machine operates as a standalone device. In some embodiments, the machine may be connected (e.g., using a network) to other machines. In a networked deployment, the machine may operate in the capacity of a server or a client user machine in server-client user network environment, or as a peer machine in a peer-to-peer (or distributed) network environment.

The machine may comprise a server computer, a client user computer, a personal computer (PC), a tablet PC, a laptop computer, a desktop computer, a control system, a network router, switch or bridge, or any machine capable of executing a set of instructions (sequential or otherwise) that specify actions to be taken by that machine. It will be understood that a device of the present disclosure includes broadly any electronic device that provides voice, video or data communication. Further, while a single machine is illustrated, the term “machine” shall also be taken to include any collection of machines that individually or jointly execute a set (or multiple sets) of instructions to perform any one or more of the methodologies discussed herein.

The computer system 800 may include a processor 802 (e.g., a central processing unit (CPU), a graphics processing unit (GPU, or both), a main memory 804 and a static memory 806, which communicate with each other via a bus 808. The computer system 800 may further include a video display unit 810 (e.g., a liquid crystal display (LCD), a flat panel, a solid state display, or a cathode ray tube (CRT)). The computer system 800 may include an input device 812 (e.g., a keyboard), a cursor control device 814 (e.g., a mouse), a disk drive unit 816, a signal generation device 818 (e.g., a speaker or remote control) and a network interface device 820.

The disk drive unit 816 may include a machine-readable medium 822 on which is stored one or more sets of instructions (e.g., software 824) embodying any one or more of the methodologies or functions described herein, including those methods illustrated above. The instructions 824 may also reside, completely or at least partially, within the main memory 804, the static memory 806, and/or within the processor 802 during execution thereof by the computer system 800. The main memory 804 and the processor 802 also may constitute machine-readable media.

Dedicated hardware implementations including, but not limited to, application specific integrated circuits, programmable logic arrays and other hardware devices can likewise be constructed to implement the methods described herein. Applications that may include the apparatus and systems of various embodiments broadly include a variety of electronic and computer systems. Some embodiments implement functions in two or more specific interconnected hardware modules or devices with related control and data signals communicated between and through the modules, or as portions of an application-specific integrated circuit. Thus, the example system is applicable to software, firmware, and hardware implementations.

In accordance with various embodiments of the present disclosure, the methods described herein are intended for operation as software programs running on a computer processor. Furthermore, software implementations can include, but not limited to, distributed processing or component/object distributed processing, parallel processing, or virtual machine processing can also be constructed to implement the methods described herein.

The present disclosure contemplates a machine readable medium containing instructions 824, or that which receives and executes instructions 824 from a propagated signal so that a device connected to a network environment 826 can send or receive voice, video or data, and to communicate over the network 826 using the instructions 824. The instructions 824 may further be transmitted or received over a network 826 via the network interface device 820.

While the machine-readable medium 822 is shown in an example embodiment to be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present disclosure.

The term “machine-readable medium” shall accordingly be taken to include, but not be limited to: solid-state memories such as a memory card or other package that houses one or more read-only (non-volatile) memories, random access memories, or other re-writable (volatile) memories; magneto-optical or optical medium such as a disk or tape; and carrier wave signals such as a signal embodying computer instructions in a transmission medium; and/or a digital file attachment to e-mail or other self-contained information archive or set of archives is considered a distribution medium equivalent to a tangible storage medium. Accordingly, the disclosure is considered to include any one or more of a machine-readable medium or a distribution medium, as listed herein and including art-recognized equivalents and successor media, in which the software implementations herein are stored.

Although the present specification describes components and functions implemented in the embodiments with reference to particular standards and protocols, the disclosure is not limited to such standards and protocols. Each of the standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same functions are considered equivalents.

The illustrations of embodiments described herein are intended to provide a general understanding of the structure of various embodiments, and they are not intended to serve as a complete description of all the elements and features of apparatus and systems that might make use of the structures described herein. Many other embodiments will be apparent to those of skill in the art upon reviewing the above description. Other embodiments may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. A system comprising:
an implantable device having a major surface interior to an organism; and
at least one biological sensor coupled to the implanted device where the biological sensor is exposed to the interior of the organism to detect a presence of bacteria and other infecting organisms in proximity to the implantable device post-operatively after the device is implanted.

2. The system of claim 1 where the at least one biological sensor outputs a signal when bacteria and other infecting organisms are detected.

3. The system of claim 2 where the implantable device is coupled to the skeletal system.

4. The system of claim 3 where the orthopedic device comprises a portion of a joint of the skeletal system.

5. The system of claim 4 where the at least one biological sensor is directed to a joint region of the implanted orthopedic device.

6. The system of claim 5 where the at least one biological sensor further comprises:
a power source;
a control circuit coupled to the power source where the control circuit includes at least one output to provide data corresponding to an output of the sensor;
a sensor operatively coupled to the control circuit where the sensor is exposed to the interior of the organism; and
a housing to protect circuitry from the interior of the organism.

7. The system of claim 4 where the at least one biological sensor detects one of pH, temperature, viscosity, or blood flow and where a measurement outside a predetermined range indicates bacteria or an infecting organism.

8. The system of claim 4 where the at least one biological sensor sends a signal through a medium in the joint region and where a change in a frequency of the signal outside a predetermined range indicates bacteria or an infecting organism.

9. The system of claim 4 where the at least one biological sensor detects cell wall markers to determine presence of bacteria or an infecting organism.

10. A system comprising:
an implantable device having a major surface interior to an organism; and
a first electrode;
a second electrode where a portion of the interior of the organism is between the first and second electrodes; and
a pulsing circuit operatively coupled to the first and second electrode where each pulse from the pulsing circuit generates an electric field between the first and second electrodes which results in electroporation of bacteria or an infecting organism in proximity to the generated electric field.

11. The system of claim 10 further including at least one biological sensor in an interior of the organism where the biological sensor detects a presence of bacteria and other infecting organisms in proximity to the implantable device after the device is implanted.

12. The system of claim 11 further including at least one biological sensor that detects one of pH, temperature, viscosity, or blood flow and where a measurement outside a predetermined range indicates bacteria or an infecting organism.

13. The system of claim 11 where the at least one biological sensor sends a signal through a medium in the joint region and where a change in a frequency of the signal outside a predetermined range indicates bacteria or an infecting organisms.

14. The system of claim 11 where the at least one biological sensor detects cell wall markers to determine presence of bacteria or an infecting organisms.

15. The system of claim 11 where the implantable device is an orthopedic device coupled to a skeletal system.

16. The system of claim 15 where the orthopedic device is an implantable joint of the skeletal system comprising one of a knee, hip, shoulder, spine, wrist, ankle, and other articulating structures of the skeletal system.

17. The system of claim 11 further including a coating comprising nanostructures on at least a portion of the major surface of the implantable device where the nanostructures house one of hydrogels, antibiotics, cytotoxins, or other medium harmful to the bacteria or other infecting organisms.

18. The system of claim 17 where the nanostructures are enabled to expose one of the hydrogels, antibiotics, cytotoxins to the bacteria or the other infecting organisms after the cell walls are made more permeable by electroporation.

19. The system of claim 11 further including a coating comprising nanostructures on at least a portion of the major surface of the implantable device where the nanostructures are enabled to attract bacteria or other infecting organisms, where the attracted bacteria or other infecting organisms enter through an opening in the nanostructures, and where the nanostructures contains the bacteria or other infecting organisms.

20. The system of claim 11 where the pulsing circuit pulses at a predetermined frequency corresponding to a resonance frequency of the bacteria or infecting organisms where a cell wall is damaged by resonance or the bacteria or infecting organisms are killed by resonance.

21. A system comprising:
an orthopedic joint implant having at least one major surface interior to an organism where a portion of the major surface has a plurality of nanostructures coupled thereto and where the nanostructures include agents to reduce infection by bacteria or an infecting organism;
at least one biosensor to detect a presence of bacteria or infecting organisms; and
a control circuit operatively coupled to the at least one biosensor and the nanostructures to enable a release of the agents contained in the nanostructures.

22. The system of claim 21 where one of hydrogels, antibiotics, or cytoxins are released from the plurality of nanostructures to kill bacteria or infecting organisms over a period time after surgically implanting the orthopedic joint to prevent bacterial growth in a joint region.

23. The system of claim 22 where a pulsed electric field is generated in proximity to the major surface of the joint implant to induce electroporation in the bacteria or infecting organisms.

24. The system of claim 21 where the presence of bacteria or infecting organisms is detected using one of pH, temperature, viscosity, blood flow, frequency change, and cell wall marker detection.

25. The system of claim 21 where a pulsed electric field is generated in proximity to the major surface of the joint implant at a resonant frequency of the bacteria or infecting organisms.
